SEQUENCE LISTING (16 sequences)

SEQ ID NO: 1 - DNA, 6701 bp, Artificial Sequence
  pMPBase Plasmid encoding piggybac transposase
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagccaccatgggcagcagcctggacgacgagcacatcctgagcgccctgctgcagagcgacgacgagctggtcggcgaggacagcgacagcgagatcagcgaccacgtgagcgaggacgacgtgcagtccgacaccgaggaggccttcatcgacgaggtgcacgaggtgcagcctaccagcagcggctccgagatcctggacgagcagaacgtgatcgagcagcccggcagctccctggccagcaacaggatcctgaccctgccccagaggaccatcaggggcaagaacaagcactgctggtccacctccaagagcaccaggcggagcagggtgtccgccctgaacatcgtgagaagccagaggggccccaccaggatgtgcaggaacatctacgaccccctgctgtgcttcaagctgttcttcaccgacgagatcatcagcgagatcgtgaagtggaccaacgccgagatcagcctgaagaggcgggagagcatgaccggcgccaccttcagggacaccaacgaggacgagatctacgccttcttcggcatcctggtgatgaccgccgtgaggaaggacaaccacatgagcaccgacgacctgttcgacagatccctgagcatggtgtacgtgagcgtgatgagcagggacagattcgacttcctgatcagatgcctgaggatggacgacaagagcatcaggcccaccctgcgggagaacgacgtgttcacccccgtgagaaagatctgggacctgttcatccaccagtgcatccagaactacacccctggcgcccacctgaccatcgacgagcagctgctgggcttcaggggcaggtgccccttcaggatgtatatccccaacaagcccagcaagtacggcatcaagatcctgatgatgtgcgacagcggcaccaagtacatgatcaacggcatgccctacctgggcaggggcacccagaccaacggcgtgcccctgggcgagtactacgtgaaggagctgtccaagcccgtccacggcagctgcagaaacatcacctgcgacaactggttcaccagcatccccctggccaagaacctgctgcaggagccctacaagctgaccatcgtgggcaccgtgagaagcaacaagagagagatccccgaggtcctgaagaacagcaggtccaggcccgtgggcaccagcatgttctgcttcgacggccccctgaccctggtgtcctacaagcccaagcccgccaagatggtgtacctgctgtccagctgcgacgaggacgccagcatcaacgagagcaccggcaagccccagatggtgatgtactacaaccagaccaagggcggcgtggacaccctggaccagatgtgcagcgtgatgacctgcagcagaaagaccaacaggtggcccatggccctgctgtacggcatgatcaacatcgcctgcatcaacagcttcatcatctacagccacaacgtgagcagcaagggcgagaaggtgcagagccggaaaaagttcatgcggaacctgtacatgagcctgacctccagcttcatgaggaagaggctggaggcccccaccctgaagagatacctgagggacaacatcagcaacatcctgcccaacgaggtgcccggcaccagcgacgacagcaccgaggagcccgtgatgaagaagaggacctactgcacctactgtcccagcaagatcagaagaaaggccaacgccagctgcaagaagtgtaagaaggtcatctgccgggagcacaacatcgacatgtgccagagctgtttctgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 2 - protein, 594 aa, Artificial Sequence
  Synthetic Construct
MGSSLDDEHILSALLQSDDELVGEDSDSEISDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTGATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRMYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPNEVPGTSDDSTEEPVMKKRTYCTYCPSKIRRKANASCKKCKKVICREHNIDMCQSCF

SEQ ID NO: 3 - DNA, 6662 bp, Artificial Sequence
  pMP-PB-HC Plasmid DNA encoding heavy chain of a Rhesus Dantibody
ggcgcgccttaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatcatcgataacaggaaagttccattggagccaagtacattgagtcaatagggactttccaatgggttttgcccagtacataaggtcaatgggaggtaagccaatgggtttttcccattactggcacgtatactgagtcattagggactttccaatgggttttgcccagtacataaggtcaataggggtgaatcaacaggaaagtcccattggagccaagtacactgagtcaatagggactttccattgggttttgcccagtacaaaaggtcaatagggggtgagtcaatgggtttttcccattattggcacgtacataaggtcaataggggtgagtcattgggtttttccagccaatttaattaaaacgccatgtactttcccaccattgacgtcaatgggctattgaaactaatgcaacgtgacctttaaacggtactttcccatagctgattaatgggaaagtaccgttctcgagccaatacacgtcaatgggaagtgaaagggcagccaaaacgtaacaccgccccggttttccctggaaattccatattggcacgcattctattggctgagctgcgttcacgtgggtataagaggcgcgaccagcgtcggtaccgtcgcagtcttcggtctgaccaccgtagaacgcagagctcctcgctgcaggcatgcaagcttggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcaaggccttgcgcttaaggagccccttcgccttttgcttgagttgaggcctggcctgggcgctggggccgccgcgtgcaaatctggtggcaccttcgcgcctgtctcgctgctttcgataagtctctagccatttaaaatttttgatgacctgctgcgacgctttttttctggcaagatantcttgtaaatgcgggccaagatctgcacactggtatttcggtttttggggccgcgggcggctacggggcccgtgcgtcccagcgcacatgttcggcgaggaggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcagggatccactagtaacggccgccagtgtgctggaattcaccatggcttgggtgtggaccttgccattcctgatggcagctgcccaaggtgtcgacgcacaggtgaaactgctcgagtctgggggaggcgtggtccagccgggggggtccctgagactctcctgtgaagcgtctggattcgccctcagaagttctggcatgcactgggtccgccaggctcctggcaaggggctggagtgggtggcacttatatggtttgatggaagtatcagatcgtatgcagaatccgtgaagggccgattcaccatctccagagacacttccaagaacaccctatatctccaaatgcgcagtctgagtgccgacgacacggctgtgtattactgtgcgagagacaaggcggttcggggaattagcaggtacaactattacatggacgtctggggcaaagggaccacggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatgaaatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagtggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatcatagctcacgctgtaggtatctcagttcggtgtaggtcgttcgctccaagctgggctgtgtgcacgaaccccccgttcagcccgaccgctgcgccttattgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaacttgtttattgcagcttataatggttacaaataaagcaatagcatcacaaatttcacaaataaagcatttttttcactgcattctagttgtggtttgtccaaactcatcaatgtatcttatcatgtctggatccgcttcaggcaccgggcttgcgggtcatgcaccaggtgcgcggtccttcgggcacctcgacgtcggcggtgacggtgaagccgagccgctcgtagaaggggaggttgcggggcgcggaggtctccaggaaggcgggcaccccggcgcgctcggccgcctccactccggggagcacgacggcgctgcccagacccttgccctggtggtcgggcgagacgccgacggtggccaggaaccacgcgggctccttgggccggtgcggcgccaggaggccttccatctgttgctgcgcggccagcctggaaccgctcaactcggccatgcgcgggccgatctcggcgaacaccgcccccgcttcgacgctctccggcgtggtccagaccgccaccgcggcgccgtcgtccgcgacccacaccttgccgatgtcgagcccgacgcgcgtgaggaagagttcttgcagctcggtgacccgctcgatgtggcggtccgggtcgacggtgtggcgcgtggcggggtagtcggcgaacgcggcggcgagggtgcgtacggcccgggggacgtcgtcgcgggtggcgaggcgcaccgtgggcttgtactcggtcatggtggcctgcagagtcgctctgtgttcgaggccacacgcgtcaccttaatatgcgaagtggacctgggaccgcgccgccccgactgcatctgcgtgttttcgccaatgacaagacgctgggcggggtttgtgtcatcatagaactaaagacatgcaaatatatttcttccggggacaccgccagcaaacgcgagcaacgggccacggggatgaagcagctggctagctaaaagttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaattcgaacagctggttctttccgcctcaggactcttcctttttcaataaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaat

SEQ ID NO: 4 - protein, 474 aa, Artificial Sequence
  Synthetic Construct
MAWVWTLPFLMAAAQGVDAQVKLLESGGGVVQPGGSLRLSCEASGFALRSSGMHWVRQAPGKGLEWVALIWFDGSIRSYAESVKGRFTISRDTSKNTLYLQMRSLSADDTAVYYCARDKAVRGISRYNYYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 200 aa, Artificial Sequence
  Synthetic Construct
SGTGLAGHAPGARSFGHLDVGGDGEAEPLVEGEVAGRGGLQEGGHPGALGRLHSGEHDGAAQTLALVVGRDADGGQEPRGLLGPVRRQEAFHLLLRGQPGTAQLGHARADLGEHRPRFDALRRGPDRHRGAVVRDPHLADVEPDAREEEFLQLGDPLDVAVRVDGVARGGVVGERGGEGAYGPGDVVAGGEAHRGLVLGH

SEQ ID NO: 6 - DNA, 6614 bp, Artificial Sequence
  pMG-PB-LC Plasmid DNA enconding light chain of Rhesus D antibody
ggcgcgccttaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatcatcgataacaggaaagttccattggagccaagtacattgagtcaatagggactttccaatgggttttgcccagtacataaggtcaatgggaggtaagccaatgggtttttcccattactggcacgtatactgagtcattagggactttccaatgggttttgcccagtacataaggtcaataggggtgaatcaacaggaaagtcccattggagccaagtacactgagtcaatagggactttccattgggttttgcccagtacaaaaggtcaatagggggtgagtcaatgggtttttcccattattggcacgtacataaggtcaataggggtgagtcattgggtttttccagccaatttaattaaaacgccatgtactttcccaccattgacgtcaatgggctattgaaactaatgcaacgtgacctttaaacggtactttcccatagctgattaatgggaaagtaccgttctcgagccaatacacgtcaatgggaagtgaaagggcagccaaaacgtaacaccgccccggttttccctggaaattccatattggcacgcattctattggctgagctgcgttcacgtgggtataagaggcgcgaccagcgtcggtaccgtcgcagtcttcggtctgaccaccgtagaacgcagagctcctcgctgcaggcatgcaagcttggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcaaggccttgcgcttaaggagccccttcgccttttgcttgagttgaggcctggcctgggcgctggggccgccgcgtgcaaatctggtggcaccttcgcgcctgtctcgctgctttcgataagtctctagccatttaaaatttttgatgacctgctgcgacgctttttttctggcaagatantcttgtaaatgcgggccaagatctgcacactggtatttcggtttttggggccgcgggcggctacggggcccgtgcgtcccagcgcacatgttcggcgaggaggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcagggatccactagtaacggccgccagtgtgctggaattcaccatgagtgtgctcactcaggtcctggcgttgctgctgctgtggcttacaggtacgcgttgtgacatcgtgatgacccagtctccatcctccctgtctgcatctgtaggagacagagtcaccatcacttgccgggcaagtcagagcattatcagatatttaaattggtatcagcacaaaccagggaaagcccctaagctcctgatccatactgcatccagtttgcaaagtggggtcccgtcaaggttcagtggcagtgtatctgggacagatttcactctcaccatcagcagtctgcaacctgaagattttgcaacttactactgtcaacagagttacactaccccgtacacttttggccaggggaccaagcttgaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcngggcctgagctcgcccgtcacaaagagcttcaacaggggagagtgttaaatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagtggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatcatagctcacgctgtaggtatctcagttcggtgtaggtcgttcgctccaagctgggctgtgtgcacgaaccccccgttcagcccgaccgctgcgccttattgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaacttgtttattgcagcttataatggttacaaataaagcaatagcatcacaaatttcacaaataaagcatttttttcactgcattctagttgtggtttgtccaaactcatcaatgtatcttatcatgtctggatccaagcttattacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggcggccgcctggacacctgtggagagaaaggcaaagtggatgtcagtaagaccaataggtgcctatcagaaacgcaagagtcttctctgtctcgacaagcccagtttctattggtctccttaaacctgtcttgtaaccttgatacttacctgcccagtgcctcacgaccaacttctgcaggatctgacggttcactaaaccagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagctagctaaaagttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaattcgaacagctggttctttccgcctcaggactcttcctttttcaataaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaat

SEQ ID NO: 7 - protein, 234 aa, Artificial Sequence
  misc_feature (219)..(219): The ′Xaa′ at location 219 stands for Gln, Arg, Pro, or Leu.
MSVLTQVLALLLLWLTGTRCDIVMTQSPSSLSASVGDRVTITCRASQSIIRYLNWYQHKPGKAPKLLIHTASSLQSGVPSRFSGSVSGTDFTLTISSLQPEDFATYYCQQSYTTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHXGLSSPVTKSFNRGEC

SEQ ID NO: 8 - protein, 240 aa, Artificial Sequence
  Synthetic Construct
LLVQLVHAESDPGGGHELQQDHVIALLVGVFAQGGLGAQVVVVGQQHGAVADGGVLLVVVGELHAAVLDVVADLEVHLDAVLLLVGHDIDVVAVVVVLQLVPQDVAVLLEVDALQLDAVHQGVALELHLGAGLVVAVVLEEDGALLDVAFGHGGLEEVVLLHVVGVAAEALHAVGQGGHEGGPGHGQLAGGADELQGQLAVGGIALALAGHAELVAVYVAVQLDQDGHHPGEQLLALAHH

SEQ ID NO: 9 - DNA, 6605 bp, Artificial Sequence
  pMP-PB-TNFR Plasmid encoding fusion protein between IgG-Fc and soluble TNF-alpha receptor
ggcgcgccttaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatcatcgataacaggaaagttccattggagccaagtacattgagtcaatagggactttccaatgggttttgcccagtacataaggtcaatgggaggtaagccaatgggtttttcccattactggcacgtatactgagtcattagggactttccaatgggttttgcccagtacataaggtcaataggggtgaatcaacaggaaagtcccattggagccaagtacactgagtcaatagggactttccattgggttttgcccagtacaaaaggtcaatagggggtgagtcaatgggtttttcccattattggcacgtacataaggtcaataggggtgagtcattgggtttttccagccaatttaattaaaacgccatgtactttcccaccattgacgtcaatgggctattgaaactaatgcaacgtgacctttaaacggtactttcccatagctgattaatgggaaagtaccgttctcgagccaatacacgtcaatgggaagtgaaagggcagccaaaacgtaacaccgccccggttttccctggaaattccatattggcacgcattctattggctgagctgcgttcacgtgggtataagaggcgcgaccagcgtcggtaccgtcgcagtcttcggtctgaccaccgtagaacgcagagctcctcgctgcaggcatgcaagcttggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcaaggccttgcgcttaaggagccccttcgccttttgcttgagttgaggcctggcctgggcgctggggccgccgcgtgcaaatctggtggcaccttcgcgcctgtctcgctgctttcgataagtctctagccatttaaaatttttgatgacctgctgcgacgctttttttctggcaagatantcttgtaaatgcgggccaagatctgcacactggtatttcggtttttggggccgcgggcggctacggggcccgtgcgtcccagcgcacatgttcggcgaggaggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcagggatccactagtaacggccgccagtgtgctggaattctgcagatatccatcacactggcggccgccatggcgcccgtcgccgtctgggccgcgctggccgtcggactggagctctgggctgcggcgcacgccttgcccgcccaggtggcatttacaccctacgccccggagcccgggagcacatgccggctcagagaatactatgaccagacagctcagatgtgctgcagcaaatgctcgccgggccaacatgcaaaagtcttctgtaccaagacctcggacaccgtgtgtgactcctgtgaggacagcacatacacccagctctggaactgggttcccgagtgcttgagctgtggctcccgctgtagctctgaccaggtggaaactcaagcctgcactcgggaacagaaccgcatctgcacctgcaggcccggctggtactgcgcgctgagcaagcaggaggggtgccggctgtgcgcgccgctgcgcaagtgccgcccgggcttcggcgtggccagaccaggaactgaaacatcagacgtggtgtgcaagccctgtgccccggggacgttctccaacacgacttcatccacggatatttgcaggccccaccagatctgtaacgtggtggccatccctgggaatgcaagcatggatgcagtctgcacgtccacgtcccccacccggagtatggccccaggggcagtacacttaccccagccagtgtccacacgatcccaactcgacgttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatgataagcttggatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagtggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatcatagctcacgctgtaggtatctcagttcggtgtaggtcgttcgctccaagctgggctgtgtgcacgaaccccccgttcagcccgaccgctgcgccttattgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaacttgtttattgcagcttataatggttacaaataaagcaatagcatcacaaatttcacaaataaagcatttttttcactgcattctagttgtggtttgtccaaactcatcaatgtatcttatcatgtctggatccgcttcaggcaccgggcttgcgggtcatgcaccaggtgcgcggtccttcgggcacctcgacgtcggcggtgacggtgaagccgagccgctcgtagaaggggaggttgcggggcgcggaggtctccaggaaggcgggcaccccggcgcgctcggccgcctccactccggggagcacgacggcgctgcccagacccttgccctggtggtcgggcgagacgccgacggtggccaggaaccacgcgggctccttgggccggtgcggcgccaggaggccttccatctgttgctgcgcggccagcctggaaccgctcaactcggccatgcgcgggccgatctcggcgaacaccgcccccgcttcgacgctctccggcgtggtccagaccgccaccgcggcgccgtcgtccgcgacccacaccttgccgatgtcgagcccgacgcgcgtgaggaagagttcttgcagctcggtgacccgctcgatgtggcggtccgggtcgacggtgtggcgcgtggcggggtagtcggcgaacgcggcggcgagggtgcgtacggcccgggggacgtcgtcgcgggtggcgaggcgcaccgtgggcttgtactcggtcatggtggcctgcagagtcgctctgtgttcgaggccacacgcgtcaccttaatatgcgaagtggacctgggaccgcgccgccccgactgcatctgcgtgttttcgccaatgacaagacgctgggcggggtttgtgtcatcatagaactaaagacatgcaaatatatttcttccggggacaccgccagcaaacgcgagcaacgggccacggggatgaagcagctggctagctaaaagttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaattcgaacagctggttctttccgcctcaggactcttcctttttcaataaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaat

SEQ ID NO: 10 - protein, 460 aa, Artificial Sequence
  Synthetic Construct
MAPVAVWAALAVGLELWAAAHALPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQLDVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - DNA, 4733 bp, Artificial Sequence
  pEGFP-N1 Commercially available plasmid expressing EGFP
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggactcagatctcgagctcaagcttcgaattctgcagtcgacggtaccgcgggcccgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaggcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaagatcgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgccatgcat

SEQ ID NO: 12 - DNA, 5166 bp, Artificial Sequence
  pSec-Tag-A Plasmid DNA
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagccaccatggagacagacacactcctgctatgggtactgctgctctgggttccaggttccactggtgacgcggcccagccggccaggcgcgccgtacgaagcttggtaccgagctcggatccactagtccagtgtggtggaattctgcagatatccagcacagtggcggccgctcgagtctagagggcccgaacaaaaactcatctcagaagaggatctgaatagcgccgtcgaccatcatcatcatcatcattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 13 - DNA, 5945 bp, Artificial Sequence
  pMG-PB Plasmid expressing EGFP
ggcgcgccttaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatcatcgataacaggaaagttccattggagccaagtacattgagtcaatagggactttccaatgggttttgcccagtacataaggtcaatgggaggtaagccaatgggtttttcccattactggcacgtatactgagtcattagggactttccaatgggttttgcccagtacataaggtcaataggggtgaatcaacaggaaagtcccattggagccaagtacactgagtcaatagggactttccattgggttttgcccagtacaaaaggtcaatagggggtgagtcaatgggtttttcccattattggcacgtacataaggtcaataggggtgagtcattgggtttttccagccaatttaattaaaacgccatgtactttcccaccattgacgtcaatgggctattgaaactaatgcaacgtgacctttaaacggtactttcccatagctgattaatgggaaagtaccgttctcgagccaatacacgtcaatgggaagtgaaagggcagccaaaacgtaacaccgccccggttttccctggaaattccatattggcacgcattctattggctgagctgcgttcacgtgggtataagaggcgcgaccagcgtcggtaccgtcgcagtcttcggtctgaccaccgtagaacgcagagctcctcgctgcaggcatgcaagcttggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcaaggccttgcgcttaaggagccccttcgccttttgcttgagttgaggcctggcctgggcgctggggccgccgcgtgcaaatctggtggcaccttcgcgcctgtctcgctgctttcgataagtctctagccatttaaaatttttgatgacctgctgcgacgctttttttctggcaagatantcttgtaaatgcgggccaagatctgcacactggtatttcggtttttggggccgcgggcggctacggggcccgtgcgtcccagcgcacatgttcggcgaggaggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcagggatccactagtaacggccgccagtgtgctggaattctgcagatatccatcacactggcggccgctcgagcatgcatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagtggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaacttgtttattgcagcttataatggttacaaataaagcaatagcatcacaaatttcacaaataaagcatttttttcactgcattctagttgtggtttgtccaaactcatcaatgtatcttatcatgtctggatccaagcttattacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggcggccgcctggacacctgtggagagaaaggcaaagtggatgtcagtaagaccaataggtgcctatcagaaacgcaagagtcttctctgtctcgacaagcccagtttctattggtctccttaaacctgtcttgtaaccttgatacttacctgcccagtgcctcacgaccaacttctgcaggatctgacggttcactaaaccagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagctagcctaaaagttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaattcgaacagctggttctttccgcctcaggactcttcctttttcaataaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaat

SEQ ID NO: 14 - protein, 240 aa, Artificial Sequence; identical to SEQ ID NO: 8
  Synthetic Construct

SEQ ID NO: 15 - DNA, 5271 bp, Artificial Sequence
  pMP-PB Plasmid encoding puromycin resistance gene
ggcgcgccttaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatcatcgataacaggaaagttccattggagccaagtacattgagtcaatagggactttccaatgggttttgcccagtacataaggtcaatgggaggtaagccaatgggtttttcccattactggcacgtatactgagtcattagggactttccaatgggttttgcccagtacataaggtcaataggggtgaatcaacaggaaagtcccattggagccaagtacactgagtcaatagggactttccattgggttttgcccagtacaaaaggtcaatagggggtgagtcaatgggtttttcccattattggcacgtacataaggtcaataggggtgagtcattgggtttttccagccaatttaattaaaacgccatgtactttcccaccattgacgtcaatgggctattgaaactaatgcaacgtgacctttaaacggtactttcccatagctgattaatgggaaagtaccgttctcgagccaatacacgtcaatgggaagtgaaagggcagccaaaacgtaacaccgccccggttttccctggaaattccatattggcacgcattctattggctgagctgcgttcacgtgggtataagaggcgcgaccagcgtcggtaccgtcgcagtcttcggtctgaccaccgtagaacgcagagctcctcgctgcaggcatgcaagcttggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcaaggccttgcgcttaaggagccccttcgccttttgcttgagttgaggcctggcctgggcgctggggccgccgcgtgcaaatctggtggcaccttcgcgcctgtctcgctgctttcgataagtctctagccatttaaaatttttgatgacctgctgcgacgctttttttctggcaagatantcttgtaaatgcgggccaagatctgcacactggtatttcggtttttggggccgcgggcggctacggggcccgtgcgtcccagcgcacatgttcggcgaggaggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcagggatccactagtaacggccgccagtgtgctggaattctgcagatatccatcacactggcggccgctcgagcatgcatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagtggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatcatagctcacgctgtaggtatctcagttcggtgtaggtcgttcgctccaagctgggctgtgtgcacgaaccccccgttcagcccgaccgctgcgccttattgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaacttgtttattgcagcttataatggttacaaataaagcaatagcatcacaaatttcacaaataaagcatttttttcactgcattctagttgtggtttgtccaaactcatcaatgtatcttatcatgtctggatccgcttcaggcaccgggcttgcgggtcatgcaccaggtgcgcggtccttcgggcacctcgacgtcggcggtgacggtgaagccgagccgctcgtagaaggggaggttgcggggcgcggaggtctccaggaaggcgggcaccccggcgcgctcggccgcctccactccggggagcacgacggcgctgcccagacccttgccctggtggtcgggcgagacgccgacggtggccaggaaccacgcgggctccttgggccggtgcggcgccaggaggccttccatctgttgctgcgcggccagcctggaaccgctcaactcggccatgcgcgggccgatctcggcgaacaccgcccccgcttcgacgctctccggcgtggtccagaccgccaccgcggcgccgtcgtccgcgacccacaccttgccgatgtcgagcccgacgcgcgtgaggaagagttcttgcagctcggtgacccgctcgatgtggcggtccgggtcgacggtgtggcgcgtggcggggtagtcggcgaacgcggcggcgagggtgcgtacggcccgggggacgtcgtcgcgggtggcgaggcgcaccgtgggcttgtactcggtcatggtggcctgcagagtcgctctgtgttcgaggccacacgcgtcaccttaatatgcgaagtggacctgggaccgcgccgccccgactgcatctgcgtgttttcgccaatgacaagacgctgggcggggtttgtgtcatcatagaactaaagacatgcaaatatatttcttccggggacaccgccagcaaacgcgagcaacgggccacggggatgaagcagctggctagctaaaagttttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaattcgaacagctggttctttccgcctcaggactcttcctttttcaataaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaat

SEQ ID NO: 16 - protein, 200 aa, Artificial Sequence; identical to SEQ ID NO: 5
  Synthetic Construct

(19) United States
(12) Patent Application Publication
(10) Pub. No.: US 2010/0311116 A1
(43) Pub. Date: Dec. 9, 2010

(54) FAST GENERATION OF HIGH EXPRESSION STABLE CELL LINES EXPRESSING RECOMBINANT PROTEINS UNDER MINIMAL AND SHORT-TERM SELECTIVE PRESSURE

(75) Inventors: Florian M. Wurm, Monthey (CH); Markus Hildinger, Zurich (CH); Maria De Jesus, Monthey (CH); Mattia Matasci, Lausanne (CH); David Hacker, Lausanne (CH)

Correspondence Address: MARKUS HILDINGER, Zugerstrasse 71A, Baar, 6340, CH

(73) Assignee: EXCELLGENE SA, Monthey (CH)

(21) Appl. No.: 12/478,558

(22) Filed: Jun. 4, 2009

(51) Int. Cl. C12N 15/87; C40B 50/06; C12P 21/00

(52) U.S. Cl. 435/69.1; 435/465; 506/26

(57) ABSTRACT

The present invention provides a novel method for the fast generation of high expression stable cell lines for the production of recombinant proteins with high efficacy of stable integration while using low selective pressure for only a short period of time. The method uses transiently expressed piggybac transposase to mediate stable integration of a transgene of interest flanked by the PB transposon termini.

### (b) CROSS-REFERENCE TO RELATED APPLICATIONS

not applicable

### (c) STATEMENT REGARDING FEDERALLY SPONSORED RESEARCH OR DEVELOPMENT

not applicable

### (d) THE NAMES OF THE PARTIES TO A JOINT RESEARCH AGREEMENT

not applicable

### (e) INCORPORATION-BY-REFERENCE OF MATERIAL SUBMITTED ON A COMPACT DISC

not applicable

### (f) BACKGROUND OF THE INVENTION

It must be noted that as used herein and in the appended claims, the singular forms “a” and “the” include plural referents unless the context clearly dictates otherwise. Thus, for example, reference to “a cell” or “the cell” includes a plurality (“cells” or “the cells”), and so forth. Moreover, the word “or” can either be exclusive in nature (i.e., either A or B, but not A and B together), or inclusive in nature (A or B, including A alone, B alone, but also A and B together). One of skill in the art will realize which interpretation is the most appropriate unless it is detailed by reference in the text as “either A or B” (exclusive “or”) or “and/or” (inclusive “or”).

Sequence information has been submitted via EFS. The text file submitted should serve as both the paper copy required by 37 CFR 1.821(c) and the CRF required by 37 CFR 1.821(e). Thus a statement under 37 CFR 1.821(f) (indicating that the paper copy and CRF copy of the sequence listing are identical) has not been included. Furthermore, the filer did not submit any additional copies of the sequence listing pursuant to 37 CFR 1.821(e).

A portion of the disclosure of this patent document contains material which is subject to copyright protection. The copyright owner has no objection to the facsimile reproduction by anyone of the patent document or the patent disclosure as it appears in the Patent and Trademark Office file or records, but otherwise reserves all copyright rights whatsoever. The patent owners can be contacted at hildinger@gmx.net.

(1) Field of the Invention

The present invention relates to a novel method for generating stable cell lines expressing recombinant proteins of interest. In that respect, the present invention relates to the field of biotechnology in general and biomanufacturing in particular.

Specifically, the present invention provides a novel method for the fast generation of stable cell lines for the production of recombinant proteins with high efficacy of stable integration while using low selective pressure for only a short period of time. The method uses transiently expressed piggybac (PB) transposase (PBase) to mediate stable integration of a transgene of interest—flanked by the PB transposon termini.

Developing stable cell lines for the commercial production of proteins for diagnosis and therapy is a costly and time consuming process due to the high heterogeneity of stable clones and the low efficacy of stable integration. Thus, methods are desirable which allow the fast and efficient generation of high-producing stable cell lines.

The present invention will provide a new and improved method for the fast generation of pools as well as single stable cell clones and cell lines by leveraging the PB transposase.

(2) Description of Related Art Including Information Disclosed Under 37 CFR 1.97 and 1.98

The present invention improves and combines existing technologies in the field of mammalian cell culture in general and in applying transposase systems in general. The process of generating high-producing mammalian cell lines represents a major bottleneck in the production of recombinant therapeutic proteins. Conventional gene transfer methods used to produce stable cell lines entirely rely on random transgene integration, consequently the probability for transgenes to become integrated into one of the rare, highly transcribed, chromosomal regions within the host genome remains considerably low. Indeed the vast majority of transfected host cells usually produce only low levels of recombinant proteins and a large number of stably transfected cells has to be analyzed in order to isolate high producing clones.

In this invention, we present a transposon mediated gene transfer method for the generation of cell lines with enhanced expression of recombinant proteins. Our system relies on the ability of transiently expressed PB transposase (PBase) to mobilize transgenes flanked by the short PB transposon termini from a donor plasmid to the host genome. The PB transposon has been shown to be active in several mammalian cell lines including Chinese Hamster Ovary (CHO) and Human Embryonic Kidney (HEK 293) cells. Furthermore, PB preferentially mediates integration into genome regions that are actively transcribed. This property may be of particular value for the generation of stable cell lines since integration into highly transcribed regions usually correlates with enhanced and stable transgene expression.

Today, recombinant cell line generation relies on methods developed in the early 1980's. These methods follow a well-defined multistep scheme that begins with the molecular cloning of the gene of interest (GOI) in a mammalian expression vector. The GOI is then delivered into cells along with a selection gene which may be cloned into the same or different expression vector. Following DNA transfer, cells are subjected to selective conditions to recover those that have stably integrated the exogenous genes into a chromosome. Well-established selection strategies rely upon complementation of a host auxotrophy. In Chinese Hamster Ovary (CHO) cells, the two main commonly used selection systems are based on the dihydrofolate reductase (dhfr) and the glutamine synthetase (gs) genes. Selection is achieved by the introduction into cells of the GOI along with a copy of the gene that complements the auxotrophy. Cells are then cultivated in medium lacking the appropriate metabolite(s) (hypoxanthine and thymidine in the case of DHFR selection and glutamine in the case of GS selection) so that only transformed clones survive.

A common alternative to the auxotrophic selection method is the use of genes conferring resistance to antibiotics such as geneticin (G418), hygromycin B, zeocin, blasticidin, or puromycin. With this strategy, transfected cells are selected using medium containing the appropriate antibiotic. The pool of cells recovered after a selection period that usually lasts for 2-3 weeks, is highly heterogeneous in terms of specific protein productivity and cell growth. This necessitates the isolation and evaluation of single cell lines to recover few candidate production clones that possess the desired characteristics. This is usually accomplished by one or more rounds of limiting dilution in which the selected cells are transferred to multiwell plates so that on average only one cell is present per well. Each clonal cell population derived from this procedure is evaluated for the level of recombinant protein expression and the highest producers are further studied for the stability of recombinant protein production since a decrease in transgene expression over time is commonly observed in the majority of clonal cell lines.

Whereas the described procedure remains one of the standard methods to establish stably transfected CHO cell lines, the whole process is quite tedious and time consuming. Indeed methods, based on transfection of plasmid DNA, result in a low success rate in terms of the generation of high-producing, stable cell lines. Usually less than 2% of the transfected cells are recovered as recombinant cell lines, and a high percentage of these (up to 80%) do not maintain a stable protein expression level after a short time in culture. For this reason, to obtain a high-producing cell line by standard methods, it is often necessary to analyze several thousand cell lines. In an industrial setting, the whole process usually takes more than six months. This is particularly inconvenient especially when multiple candidate therapeutics need to be produced in high enough amounts to be evaluated for efficacy and safety in preclinical studies or clinical trials.

The PB transposon was discovered in an insect virus, but several studies have shown the ability of PB to actively transpose in mammalian cells. Studies describing transposition by PB transposase systems to date have been mainly focused on the generation of mutations in transgenic animals. Due to its ability to transpose in mammalian cells and promote stable transgene expression, the PB transposon has been also proposed as a tool for gene therapy. However, in this context, technical problems and safety concerns have still to be successfully addressed. In particular due to the ability of the piggyback transposase to promote integration into actively transcribed genes, piggyback mediated transgene insertion can cause genetic alterations that may ultimately results in the onset of diseases. In recent studies the piggybac-based vectors have been used to achieve concomitantly recombinant expression of the four transcription factors c-Myc, Klf4, Oct4 and Sox2 factors in mice fibroblasts with the final aim to generate induced pluripotent stem cells (iPS).

Whereas several studies have used the PB transposon to obtain stable expression of transgenes in mammalian cell lines, to our knowledge there has not yet been any publication describing its application to the generation of high expressing stable cell lines for recombinant protein manufacturing.

### (g) BRIEF SUMMARY OF THE INVENTION

(1) Substance or General Idea of the Claimed Invention

The present invention provides a method for creating a stable CHO cell line or a pool of stable CHO cell lines by co-transfecting a plasmid harboring the piggybac (PB) transposase (PBase) with a plasmid harboring a gene expression cassette flanked by piggybac minimal inverted repeat elements where the gene expression cassette flanked by piggybac minimal inverted repeat elements is stably integrated into the CHO genome whereas the plasmid harboring the piggybac transposase is not. For purposes of this invention, the term “stable cell line” and “stable cell clone” is used interchangeably.

In a further aspect, the present invention provides a method for creating a CHO cell line or a pool of stable CHO cell lines by co-transfecting a plasmid harboring a PBase expression cassette with a plasmid harboring a gene expression cassette of a protein of interest flanked by piggybac minimal inverted repeat elements where a higher number of stable CHO cell clones is obtained with PB transposase expression cassette co-transfection compared to transfection without PB transposase expression cassette co-transfection. This allows a more rapid recovery of a (multi-clonal) cell suspension, which then can be expanded to produce large quantities of the recombinant protein of interest in a short period of time. In some embodiments, such a stable pool of CHO cells can be obtained in less than 10 days.

In yet another aspect, the present invention allows the generation of a pool of stable cell clones where the expression levels of the protein of interest in the pool of stable cell clones generated using the PB transposon system are higher compared to a pool of stable cell clones generated in the absence of the PB transposon system. In some embodiments, this is due to a higher specific productivity of the stable cell clones. In other embodiments, this is due to a higher percentage of stably transfected cell clones (leading to a higher recovery rate). In yet other embodiments, this is due to both effects, i.e., higher specific productivity and a higher percentage of stably transfected clones (leading to a higher recovery rate).

In some embodiments, puromycin at a concentration of 10 mg/l is used as a selective agent. In some embodiments, puromycin selection is used for 10 days, in other embodiments, puromycin selection is used for less than 10 days.

In some embodiments, the gene expression cassette flanked by the piggybac minimal inverted repeat elements comprises the genetic information for a secreted protein. In other embodiments, said gene expression cassette comprises the genetic information for a non-secreted protein, an intracellular protein or a membrane-bound protein.

In some embodiments, the gene expression cassette flanked by the piggybac minimal inverted repeat elements comprises the genetic information for an antibody or an Fc-fusion protein, and the protein of interest is expressed at a specific productivity of at least 20 pg per cell per day.

This invention—for the first time—describes a method for the fast generation of a stable cell line and/or a pool of stable cell clones.

In addition, this invention—for the first time—describes a method for the fast production of recombinant proteins by a stable cell line and/or a pool of stable cell clones.

Similarly, this invention—for the first time—describes a method for the fast generation of a stable cell line and/or a pool of stable cell lines with high specific productivity.

(2) Advantages of the Invention Over Prior Approaches

Usefulness of the Present Invention

The present invention is useful for mammalian cell culture and biomanufacturing, both in free suspension as well for anchorage-dependent systems. A significant proportion of commercially available recombinant proteins are produced in mammalian stable cell lines in general, and CHO cells in particular. Yet, creating a stable cell line that expresses the protein of interest over an extended period of time and at high expression levels is a time intensive and costly process. Here, the present invention provides a useful advancement of the current art.

The present invention allows for the fast generation of a pool of stable clones as the process of stable integration is facilitated by the PB transposase. Thus, it will require less time to produce a pool of stable clones compared to traditional techniques, i.e., in the presence of the PB transposase, more clones are generated compared to a process that does not utilize the PB transposase. This is useful in the field of biomanufacturing as faster time to clones means time savings and thus potentially a longer commercial phase prior to patent expiry.

Furthermore, the present invention generates clones with higher specific productivity compared to traditional techniques, i.e., in the presence of the PB transposase, clones are generated with higher specific productivity compared to clones established in the absence of PB transposase. This is useful in the field of biomanufacturing as higher specific productivity means lower manufacturing cost—all else equal.

Novelty of the Present Invention

Whereas the PB transposase system has been widely used to create stable cell lines, the inventors are the first to describe the application of the PB transposase system for the generation of stable CHO cell lines for the expression of secreted recombinant proteins that are superior to stable CHO cell lines established in the absence of the PB transposase in respect to (a) the number of stable CHO clones to be generated, (b) the time it takes to establish a certain number of stable CHO clones, (c) the specific productivity, and (d) the overall product yield as a function of time after initial transfection.

Non-Obviousness of the Present Invention

The present invention combines multiple aspects in a non-obvious way to achieve an improved process for the generation of stable cell lines.

Whereas individual elements of the present invention are in the public domain, it is not obvious that the combination of those elements will yield a method that is superior to stable CHO cell lines established in the absence of the PB transposase in respect to (a) the number of stable CHO clones to be generated, (b) the time it takes to establish a certain number of stable CHO clones, (c) the specific productivity, and (d) the overall product yield as a function of time after initial transfection.

Given the high commercial interest in biomanufacturing and the significant investment of biopharmaceutical companies, it is not obvious that applying the PB transposase system to the generation of stable cell lines will yield CHO cell lines and CHO clone populations that are superior in terms of (a) the number of stable CHO clones to be generated, (b) the time it takes to establish a certain number of stable CHO clones, (c) the specific productivity, and (d) the overall product yield as a function of time after initial transfection.

### (h) BRIEF DESCRIPTION OF THE SEVERAL VIEWS OF THE DRAWING(S)

FIG. 1: Transgene expression analysis in cell populations generated by transposition (PIT, PTT) or conventional transfection (PIS, PTS)

FIG. 2: Cell specific productivity of selected clones was measured using the integral cell viability (IVC) method (Renard et al). Clones PIT-7, -23 and PTT-3; -7 expressing an IgG or a TNFR:Fc fusion protein were generated by transposition, and the corresponding control clones PIS-1, -17 and PTS-18, -22, by normal transfection.

FIG. 3: Cell specific productivity of selected clones was measured using the integral cell viability (IVC) method (Renard et al). Clones PIT-7, -23 and PTT-3; -7 expressing an IgG or a TNFR:Fc fusion protein were generated by transposition, and the corresponding control clones PIS-1, -17 and PTS-18, -22, by normal transfection. Relative transgene copy in selected clones shown.

FIG. 4: Analysis of the productivity of single clones sorted by limiting dilution from cell populations generated by transposition or conventional transfection. The supernatant of 5 days old batch cultures was analysed by ELISA.

FIG. 5: Transposition activity of piggybac in CHO-DG44 mammalian cells. A total of 2 million cells in 12 well plates were transfected with 1.25 ug of donor plasmid along with varying amounts of helper vector. The pSecTagA [SEQ-ID NO:12] vector served as filler DNA to keep the amount of DNA used for transfection constant. Following a ten days selection period transposition efficiency was measured by counting puromycin-resistant colonies. Data are shown as mean values with SD (n=4).

FIG. 6: Analysis of the cell viability during the puromycin selection process. Transposed and transfected cell were subjected cultivated in presence of 10 or 50 mg/l of puromycin. Tx, day of transfection; Puro, starting day of selection.

FIG. 7: Equations used to determines the number of independent stable clones generated at the day of transfection and the efficiency of stable cell line generation relative to the total number of transfected cells.

FIG. 8: Summary of stable cell line generation efficiency

FIG. 9: Analysis of the number of GFP expressing cells over time following transposition or transfection by GUAVA. Transposed cells were generated by cotransfecting CHO cells with the pMG-PB-TNFR [SEQ-ID NO:9] (donor) and pmPBase [SEQ-ID NO:1] (helper) vectors, transfection was achieved by cotransfecting cells with the pMG-PB-TNFR [SEQ-ID NO:9] (donor) and pSecTagA [SEQ-ID NO:12] (Invitrogen) helper devoid of the PBase gene) vectors.

FIG. 10: Percentage of the GFP positive cells in the cells populations generated by transposition or transfection during a period of 9 days.

FIG. 11: Supernatant of 5 days culture seeded into 24 wells plates were analysed by ELISA. Cell lines were classified based on TNFR:Fc expression level into four categories, very low (<4 mg/l), low (4-50 mg/l), middle (50-100 mg/l) and high (100-200 mg/l) producers.

FIG. 12: Following screening the best expressing clones sorted from transposed (black) or transfected (gray) populations that were selected either with 10 (hatched bars) or 50 (filled bars) ug/ml puromycin further expanded and cultivated into Tubespin bioreactors in order to better assess volumetric productivity. Supernatant from 4 days cultures was analysed by ELISA

### (i) DETAILED DESCRIPTION OF THE INVENTION

### (1) Definitions

In describing the present invention, the following terms will be employed, and are intended to be defined as indicated below.

For purpose of this invention, the term “protein” means a polypeptide (native [i.e., naturally-occurring] or mutant), oligopeptide, peptide, or other amino acid sequence. As used herein, “protein” is not limited to native or full-length proteins, but is meant to encompass protein fragments having a desired activity or other desirable biological characteristics, as well as mutants or derivatives of such proteins or protein fragments that retain a desired activity or other biological characteristic including peptoids with nitrogen based backbone. Mutant proteins encompass proteins having an amino acid sequence that is altered relative to the native protein from which it is derived, where the alterations can include amino acid substitutions (conservative or non-conservative), deletions, or additions (e.g., as in a fusion protein). “Protein” and “polypeptide” are used interchangeably herein without intending to limit the scope of either term.

For purposes of this invention, “amino acid” refers to a monomeric unit of a peptide, polypeptide, or protein. There are twenty amino acids found in naturally occurring peptides, polypeptides and proteins, all of which are L-isomers. The term also includes analogs of the amino acids and D-isomers of the protein amino acids and their analogs.

For purposes of this invention, by the term “transgene” is meant a nucleic acid composition made out of DNA, which encodes a peptide, oligopeptide or protein. The transgene may be operatively linked to regulatory control elements in a manner which permits transgene transcription, translation and/or ultimately directs expression of a product encoded by the expression cassette in the producer cell, e.g., the transgene is placed into operative association with a promoter and enhancer elements, as well as other regulatory control elements, such as introns or polyA sequences, useful for its regulation. The composite association of the transgene with its regulatory sequences (regulatory control elements) is referred to herein as a “minicassette”, “expression cassette”, “transgene expression cassette”, or “minigene”. The exact composition of the expression cassette will depend upon the use to which the resulting (mini)gene transfer vector will be put and is known to the artisan (Sambrook 1989, Lodish et al. 2000). When taken up by a target cell, the expression cassette as part of the recombinant vector genome may remain present in the cell as a functioning extrachromosomal molecule, or it may integrate into the cell's chromosomal DNA, depending on the kind of transfer vector used. Generally, a minigene may have a size in the range of several hundred base pairs up to about 30 kb.

For purposes of this invention, the term “cell” means any prokaryotic or eukaryotic cell, either ex vivo, in vitro or in vivo, either separate (in suspension) or as part of a higher structure such as but not limited to organs or tissues.

For purposes of this invention, the term “host cell” means a cell that can be transduced and/or transfected by an appropriate gene transfer vector. The nature of the host cell may vary from gene transfer vector to gene transfer vector.

For purposes of this invention, the term “producer cell” means a cell that is capable of producing a recombinant protein or protein of interest. The producer cell itself may be selected from any mammalian cell. Particularly desirable producer cells are selected from among any mammalian species, including, without limitation, cells such as HEK 293, A549, WEHI, 3T3, 10T1/2, BHK, MDCK, COS 1, COS 7, BSC 1, BSC 40, BMT 10, VERO, WI38, HeLa, Saos, C2C12, L cells, HT1080, HepG2, CHO, NS0, Per.C6. The selection of the mammalian species providing the cells is not a limitation of this invention; nor is the type of mammalian cell, i.e., fibroblast, hepatocyte, tumor cell, etc. Frequently used producer cells or HEK 293 cells, BHK cells, NS0 cells, Per.C6 cells and CHO cells. Preferentially, a producer cell should be free of potential adventitious viruses.

For purposes of this invention, “transfection” is used to refer to the uptake of nucleic acid compositions by a cell. A cell has been “transfected” when an exogenous nucleic acid composition has crossed the cell membrane. A number of transfection techniques are generally known in the art. Such techniques can be used to introduce one or more nucleic acid compositions, such as a plasmid vector and other nucleic acid molecules, into suitable host cells. Frequently, cells are transfected with 25-kd linear polyethyleneimine. Other alternatives are transfection by means of electroporation, liposomes, dendrimers, or calcium phosphate.

For purposes of this invention, by “vector”, “transfer vector”, “gene transfer vector” or “nucleic acid composition transfer vector” is meant any element, such as a plasmid, phage, transposon, cosmid, chromosome, virus, virus capsid, virion, etc., which is capable of transferring and/or transporting a nucleic acid composition to a host cell, into a host cell and/or to a specific location and/or compartment within a host cell. Thus, the term includes cloning and expression vehicles, as well as viral and non-viral vectors and potentially naked or complexed DNA. However, the term does not include cells that produce gene transfer vectors such as retroviral packaging cell lines.

For purpose of this invention, the term “specific productivity” refers to the amount of the protein of interest that is produced by a single cell per day. For example a specific productivity of 20 pg/cell/day refers to the production of 20 pg of the protein of interest by a single cell within 24 hours.

For purpose of this invention, the term “batch” refers to the (specific lot of) protein molecules of interest produced in a single production run, i.e., under the same production conditions. Batch means a specific quantity of a drug or other material that is intended to have uniform character and quality, within specified limits, and is produced according to a single manufacturing order during the same cycle of manufacture.

For purpose of this invention, the term “lot” means a batch, or a specific identified portion of a batch, having uniform character and quality within specified limits; or, in the case of a drug product produced by continuous process, it is a specific identified amount produced in a unit of time or quantity in a manner that assures its having uniform character and quality within specified limits

For purpose of this invention, the term “batch yield” refers to the maximum amount (in grams) of the recombinant protein of interest produced by all of the mammalian cells in the culture batch together. For secreted proteins, the “batch yield” refers to the maximum amount of the recombinant protein of interest in the culture medium where the recombinant protein of interest is secreted into the medium by the mammalian cells present in the medium. For example, if a mammalian cell culture of 1 liter comprises 0.5 g of recombinant protein of interest in total, the batch yield is 500 mg and the batch titer is 500 mg/l. Thus, whereas the specific productivity refers to the production of recombinant protein by a single mammalian cell within one day, the batch yield refers to the maximum amount of recombinant protein produced by all the mammalian cells in the culture during the total time of the culture. “Volumetric yield” can be used as a synonym for “batch yield”.

For purpose of this invention, the term “batch titer” refers to the maximum concentration (in grams per liter or milligrams per liter) of the recombinant protein of interest produced by all of the mammalian cells in the culture batch together. For secreted proteins, the “batch titer” refers to the maximum concentration of the recombinant protein of interest in the culture medium where the recombinant protein of interest is secreted into the medium by the mammalian cells present in the medium. For example, if a mammalian cell culture of 1 liter comprises 0.5 g of recombinant protein of interest in total, the batch yield is 0.5 grams and the batch titer is 0.5 g/l. Thus, whereas the specific productivity refers to the production of recombinant protein by a single mammalian cell within one day, the batch titer refers to the maximum concentration of recombinant protein produced by all the mammalian cells in the culture during the total time of the culture. The batch titer could also be defined as batch yield divided by culture volume.

For purpose of this invention, “growth medium” refers to a cell culture medium that promotes cell growth and division—leading to an increase in biomass as it relates to the cells. Optimally, a growth medium allows for a fast increase in biomass and supports cell growth to high cell densities.

For purpose of this invention, “transfection medium” refers to a cell culture medium that is suitable for transfection. Transfection media do not necessarily support cell growth or production. For example, RPMI can be used as transfection medium, but is not well suited for cell growth or production. An optimal transfection medium does not interfere with the transfection process, e.g., it does not contain inhibitors that inactivate the transfection reagent.

For purpose of this invention, “production medium” refers to a cell culture medium that promotes production of the protein of interest. A production medium does not necessarily support cell growth. Furthermore, one cannot necessarily transfect in production media, or only at a low transfection efficacy. An optimal production medium has the following characteristics: It sustains cell viability at a high cell density and results in high specific productivity for an extended period of time.

### (2) General Methods

The practice of the present invention will employ, unless otherwise indicated, conventional methods of microbiology, molecular biology and recombinant DNA techniques within the skill of the art. Such techniques are explained fully in the literature; see, e.g., Sambrook, et al. Molecular Cloning: A Laboratory Manual (Current Edition); DNA Cloning: A Practical Approach, vol. I & II (D. Glover, ed.); Oligonucleotide Synthesis (N. Gait, ed., Current Edition); Nucleic Acid Hybridization (B. Hames & S. Higgins, eds., Current Edition); Transcription and Translation (B. Hames & S. Higgins, eds., Current Edition); CRC Handbook of Parvoviruses, vol. I & II (P. Tijessen, ed.); Fundamental Virology, 2nd Edition, vol. I & II (B. N. Fields and D. M. Knipe, eds.)

### (3) Preferred Embodiment, i.e., Best Mode Contemplated by the Inventors of Carrying Out the Present Invention

Suspension-adapted CHO DG44 cells were grown in ProCHO5 medium (Lonza A G, Viège, Switzerland) supplemented with 0.68 mg/l hypoxanthine, 0.194 mg/l thymidine, and 4 mM glutamine (SAFC Biosciences, St. Louis, Mo.).

Transfections were carried out in 50-ml ventilated centrifuge tubes (CultiFlask 50 tubes; Sartorius A G, Goettingen, Germany) as previously described in Muller, N., et al., Scalable transient gene expression in Chinese hamster ovary cells in instrumented and non-instrumented cultivation systems. Biotechnol Lett, 2007. 29(5): p. 703-11. For the generation of lines expressing an IgG antibody a mixture of pMP-PB-HC [SEQ-ID NO:3], pMG-PB-LC [SEQ-ID NO:6] and pmPBase [SEQ-ID NO:1] vectors at a ratio of 1:1:2 was used. Cells expressing a TNFR:Fc fusion protein were generated using pMG-PB-TNFR [SEQ-ID NO:9] and pmPBase [SEQ-ID NO:1] vectors at a ratio of 1:1. Transfected cells were subjected to a selective pressure with 10 mg/l puromycin for 10 days, after which cells were further cultivated in absence of any selection. Two months following transfection single clones were derived using the limiting dilution technique.

The donor vectors used for the expression of a recombinant IgG antibody, (pMP-PB-HC [SEQ-ID NO:3] and pMG-PB-LC [SEQ-ID NO:6]) consist of artificial transposons carrying the heavy chain (HC) and puromycin resistance or the light chain (LC) and the enhanced green fluorescent protein (eGFP) genes respectively. For the expression of the TNFR:Fc, a donor vector (pMG-PB-TNFR [SEQ-ID NO:9]) carrying the TNFR:Fc fusion gene was used. The helper vector (pmPBase [SEQ-ID NO:1]) used for the transient expression of the transposase enzyme, carries a codon optimized variant of the PB-transposase. Control cell populations were generated with a helper vector devoided of the PBase gene.

IgG and TNFR:Fc concentration in the cell culture medium was determined by sandwich ELISA as described by Pick, H. M., et al., Balancing GFP reporter plasmid quantity in large-scale transient transfections for recombinant anti-human Rhesus-D IgG1 synthesis. Biotechnol Bioeng, 2002. 79(6): p. 595-601. Cell specific productivity was determined by plotting recombinant protein concentration values against the integral of viable cells (IVC) as described by Renard, J. M., et al., Evidence that monoclonal antibody production kinetics is related to the integral of the viable cells curve in batch systems. Biotechnology Letters, 1988. 10(2): p. 91-96.

The number of transgene copies in the single cell clones was estimated by real time PCR using primers specific to the HC, LC or TNFR:Fc transgenes and genomic DNA as template. Results were analyzed by the 2-ΔΔCT method using the β-actin gene as endogeneous control as described by Livak, K. J. and T. D. Schmittgen, Analysis of relative gene expression data using real-time quantitative PCR and the 2(-Delta Delta C(T)) Method. Methods, 2001. 25(4): p. 402-8.

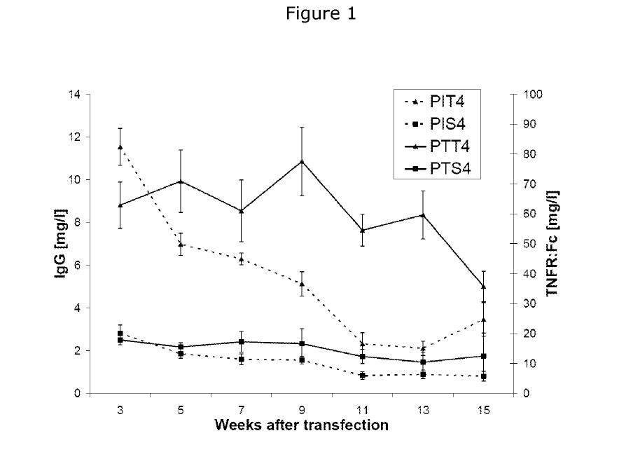

Transposed cell populations expressing an IgG antibody or a TNFR:Fc fusion protein (PIT and PTT respectively) and the corresponding control populations (PIS and PTS respectively) were generated by transfection of CHO-DG44 cells with the appropriate donor and helper plasmids. Following puromycin selection polyclonal cell populations were cultivated in absence of any selection and analysed on a weekly basis for the level of recombinant protein expression. As shown in FIG. 1 levels of IgG or TNFR:Fc expression in the PIT and PTT cell populations were on average 3.5 respectively 4.2 times higher than these of the corresponding control populations (PIS, PTS respectively). This increase was particularly evident when the TNFR:Fc was used as model protein.

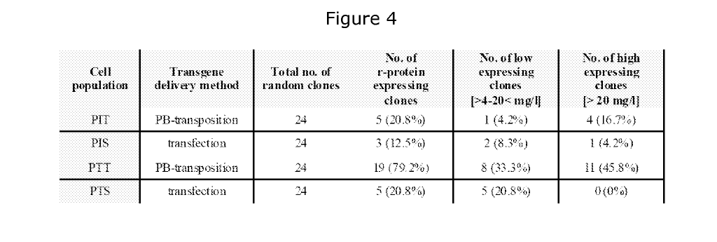

To assess whether the observed increase in transgene expression was a result of either a) a higher percentage of cells expressing the transgene b) an increased cell specific productivity of the single cells or c) a combination of these two factors, two months after transfection random clones were sorted by limiting dilution. Accordingly, from each population 24 clonal lines were recovered and analysed for productivity. As summarized in FIG. 4, transposon mediated gene delivery resulted in an improvement of both the percentage of clones expressing the transgene and the level of recombinant gene expression.

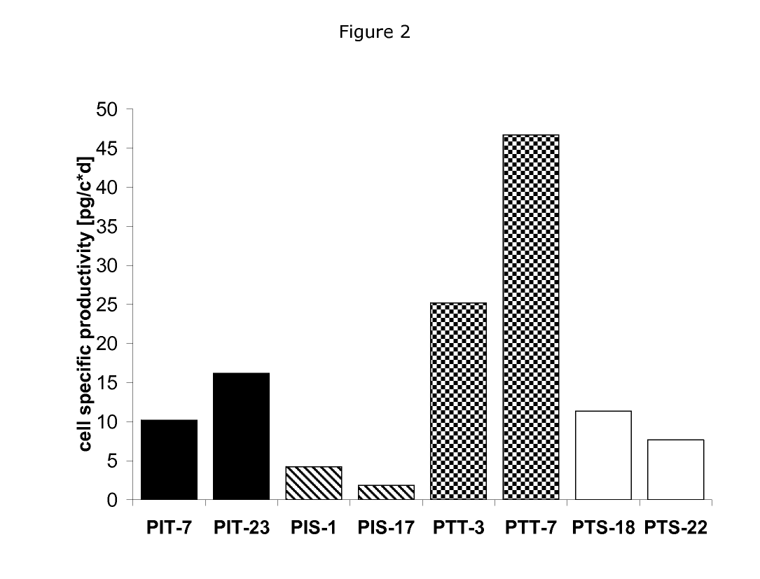

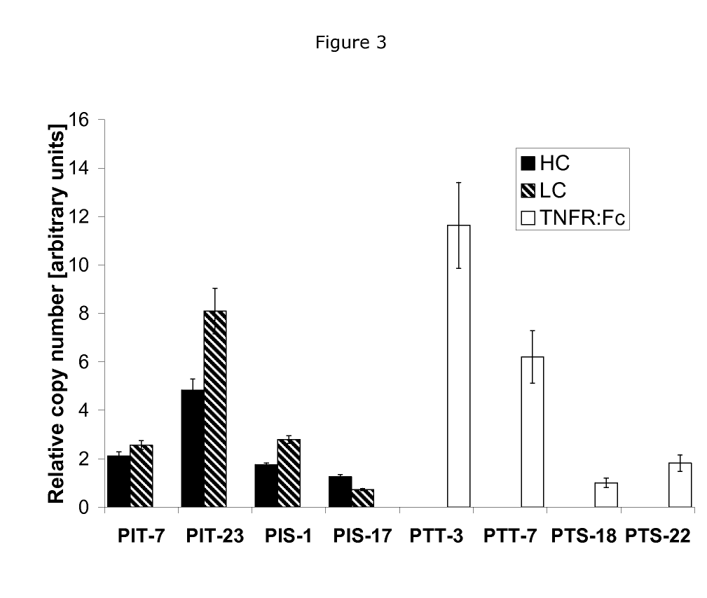

The number of clonal lines expressing detectable levels of the recombinant IgG was 1.7 times higher for clones sorted from the transposed population (5 vs. 3 clones), in addition 16.7% of clones generated by transposition showed an IgG expression level higher than 20 mg/l. Similar results were obtained for the expression of the TNFR:Fc fusion protein. In this case the overall number of clones expressing the recombinant protein was increased of 3.8 times and more than 45% of the transposed clones produced titers of TNFR:Fc levels greater than 20 mg/l. Comparison of the best performing clones obtained from the different populations confirmed a general increase of the cell specific productivity for clones generated by transposition (FIG. 2). This increased productivity in part correlated with an enhanced number of integrated transgene copies as determined by qPCR (FIG. 3).

We further verified the absence of PB transposase integration by PCR. At the conditions chosen, the PB transposase gene was not stably integrated into the clones.

Taken together these results show that upon transposition both the percentage of transgene expressing cells as well as the levels of transgene expression are improved thus demonstrating the efficiency of the PB transposon system for the generation of high producing cell lines.

### (4) Other Embodiments of the Present Invention

4.1 Generation of the pMP-PB [SEQ-ID NO:15] and pMG-PB [SEQ-ID NO:13] Donor and pmPBase [SEQ-ID NO:1] Helper Vectors.

The pMP-PB [SEQ-ID NO:15] vector carries an artificial transposon with a puromycin-resistance selection marker and a cassette for transgene expression flanked by the left and right terminal domains sequences (LTD, RTD) of the PB transposon. In the pMG-PB [SEQ-ID NO:13] vector the puromycin cassette has been replaced by a cassette for the expression of the enhanced green fluorescent protein (EGFP). The heavy and light chain cDNA were obtained by digesting the pKMH and pKML with EcoRI and NotI and cloned into pMP-PB [SEQ-ID NO:15] and pMG-PB [SEQ-ID NO:13] digested with the same enzymes to obtain the vectors pMG-PB-HC [SEQ-ID NO:3] and p-MG-PB-LC [SEQ-ID NO:6] respectively. The pMG-PB-TNFR [SEQ-ID NO:9] was constructed by subcloning a gene-synthesized TNFR:Fc cDNA as NotI BamHI fragment into pMP-PB [SEQ-ID NO:15] digested with NotI and BclII. The pmPBase [SEQ-ID NO:1] helper construct carries a codon optimized variant of the PB transposase cDNA under the control of the human CMV promoter.

The inventors have provided an electronic version of the sequence information of all the plasmids used in the present invention. Current art enables one of ordinary skill to obtain any of those plasmids through gene synthesis services based on the sequence information provided. Such services are commonly available, e.g., from GeneArt in Germany or DNA2.0 in the United States.

4.2 Cell Culture

Suspension-adapted CHO DG44 cells were grown in serum-free ProCHO5 medium (Lonza A G, Viège, Switzerland) supplemented with 0.68 mg/l hypoxanthine, 0.194 mg/l thymidine, and 4 mM glutamine (SAFC Biosciences, St. Louis, Mo.). The cells were maintained in 10 ml of medium at 37° C. in 95% humidity and 5% CO2 in 50-ml ventilated centrifuge tubes (Sartorius A G, Goettingen, Germany) with agitation at 180 rpm on a model ES-W orbital shaker (Kühner A G, Birsfelden, Switzerland). The cells were transferred to fresh medium twice per week at a density of 3e5 cells/ml

4.3 DNA Transfection

Suspension adapted CHO-DG44 cells were transfected using PEI as DNA delivery agent. The day before transfection cells were seeded in fresh medium at a density of 1e6 cells/ml. On the day of transfection cells were centrifuged and resuspended in 5 ml of ProCHO5 medium with supplements in 50-ml ventilated centrifuge tubes (TPP, Trasadingen, Switzerland) at a density of 2e6 cells/ml. Stock solutions of DNA and PEI were diluted separately in sterile 150 mM NaCl. The PEI solution was then added to the diluted DNA and the mixture was incubated at room temperature for 10 min. Finally, 500 μl of 150 mM NaCl containing 12.5 μg of DNA and 50 μg of linear 25-kDa PEI was added to each tube. The tubes were then incubated as described above. Four hours after transfection cells were diluted with fresh medium to a cell density of 1e6 cells/ml.

Following plasmid ratios were used for the generation of stable cells expressing a) EGFP: pMG-PB [SEQ-ID NO:13] 50%, pmPBase [SEQ-ID NO:1] 50%, b) an IgG-1 monoclonal antibody: pMP-PB-HC [SEQ-ID NO:3] 25%, pMG-PB-LC [SEQ-ID NO:6] 25%, pmPBase [SEQ-ID NO:1] 50%, and c) the TNFR2:Fc: pMG-PB-TNFR [SEQ-ID NO:9] 50%, pmPBase [SEQ-ID NO:1] 50%. For all transfection control populations were generated in which the pmPBase [SEQ-ID NO:1] helper vector was replaced by the pSecTagA [SEQ-ID NO:12] vector not encoding the transposase.

4.4 Recovery of Recombinant Cell Lines by Limiting Dilution

To establish populations of stably transfected cells, the day after transfection cell medium was replaced with fresh media containing 10 μg/ml puromycin. The selective pressure was maintained for 10 days, after which cells were further cultivated in absence of any selection. Two months after transfection single clones were recovered from the populations of stably transfected cells by limiting dilution. Typically, single clones were seeded in 96-well plates with 200 μl of fresh medium, and each well was checked microscopically on a daily base to confirm single cell growth.

4.5 Protein Analysis

4.5.1 GFP Expression Analysis

For quantification of GFP in suspension cultures, the cells were centrifuged and resuspended in PBS at a density of about 5e5 cells/ml. Quantification of GFP fluorescence was performed using a GuavaEasyCyte flow cytometer and Guava Express plus software (v3.6.1).

4.5.2 ELISAs

The IgG concentration in the culture medium was determined by sandwich ELISA as previously described. In short, goat anti-human kappa light chain IgG (Biosource, Dielsdorf, Switzerland) was used for coating the ELISA-plates, and the synthesized IgG1 was detected with AP-conjugated goat anti-human gamma chain IgG (Biosource, Dielsdorf, Switzerland). NPP was used as a substrate for the alkaline phosphatase. Absorption was measured at 405 nm against 490 nm using a microplate reader (SPECTRAmax™340; Molecular Devices, Palo Alto, Calif., USA).

To measure TNFR:Fc secretion anti goat anti-human IgG (Fc Fragment specific; Jackson ImmunoResearch Laboratories Inc, West Grove, Pa., USA) was used for coating the ELISA-plates. As a detection antibody, we used AP-conjugated goat anti-human gamma chain IgG (Biosource, Dielsdorf, Switzerland). Purified TNFR:Fc molecule was used as standards. Other details of the ELISA were similar to the IgG ELISA.

4.5.3 Colony Counting Assay

To assess integration efficiency, cells were harvested and seeded at 2e6 cell/ml into individual wells of 12-well plates 24 hours before transfection. The transfection cocktail contained 50% of the pMG-PB [SEQ-ID NO:13] donor plasmid plus 0 to 50% of the helper plasmid pmBPase [SEQ-ID NO:1], the total amount of transfected DNA in each assay was kept constant to 2.5 ug by adding pSecTagA [SEQ-ID NO:12] vector as filler DNA. One day after transfection cells were diluted 1:200 and seeded into 100 mm plates in DMEM-F12 medium supplemented with hypoxanthine, thymidine, glutamine, 5% FBS followed. Stable cells were selected in the presence of 10 μg/ml puromycin for 10 days. To count the colonies, cells were washed with PBS and stained with 1% methylene blue in 50% methanol for 30 min.

4.6 Results

4.6.1 Piggybac Transposition Enhances Stable Transgene Integration in CHO-DG44 Cells

To assess efficiency of PB-mediated transgenesis in suspension adapted CHO-DG44 cells, we performed a series of experiments in which cells were co-transfected with a donor plasmid, pMG-PB [SEQ-ID NO:13], carrying the puromycin and EGFP genes flanked by PB terminal repeats, along with a pmPBase [SEQ-ID NO:1] helper plasmid containing a codon optimized variant of the PB transposase driven by the CMV promoter. To address saturation effect the amount of the pMG-PB [SEQ-ID NO:13] vector was keep constant whereas the amount of the pmPBase [SEQ-ID NO:1] plasmid was varied from 0 to 50% of the total amount of DNA used for transfection. (pMG-PB and pMG-PB-eGFP are used synonymously for the purpose of this invention). Resistant colonies were counted after 10 days of 10 ug/ml of puromycin selection.

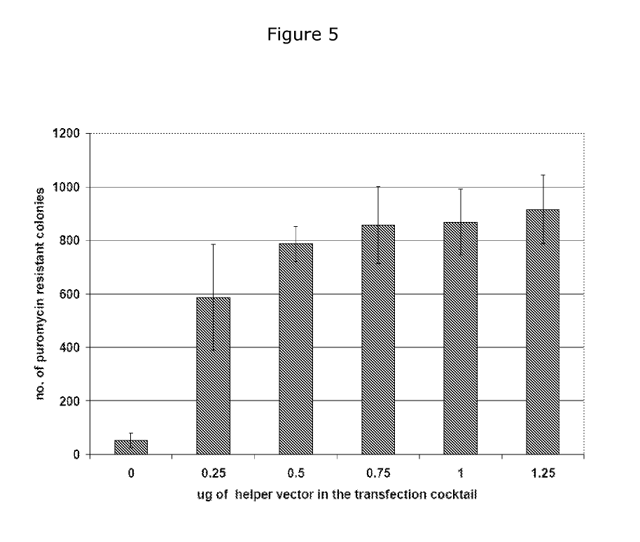

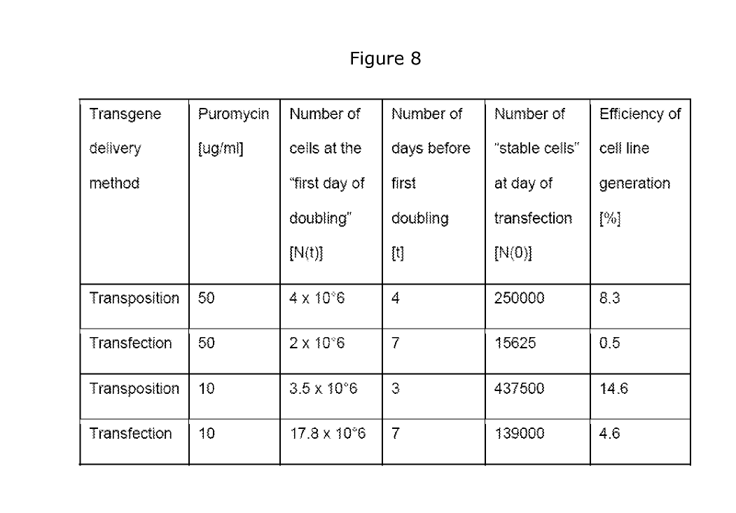

As shown in FIG. 5, in all conditions tested, cells co-transfected with the pmPBase [SEQ-ID NO:1] helper vector resulted in considerably more puromycin resistant colonies than control cells co-transfected with the pSecTagA [SEQ-ID NO:12] vector. Furthermore in our experiments we did not observe overproduction inhibition, a phenomenon that has been described in certain transposon systems in which efficiency of transposition is reduced when the amount of transposase exceeds certain levels. As indicated in FIG. 8, when compared to control transfections done in absence of the transposase, PB transposition resulted in an 11 to 17 folds increased efficiency of transgenesis that corresponded to a 3 to 5% rate of stable transgene integration. These results on the efficiency of stable transgene integration upon transposition in CHO cells are in line with efficiencies reported by other groups.

4.6.2 Transposed Cell Populations Recover Faster from Puromycin Selection than Transfected Cells

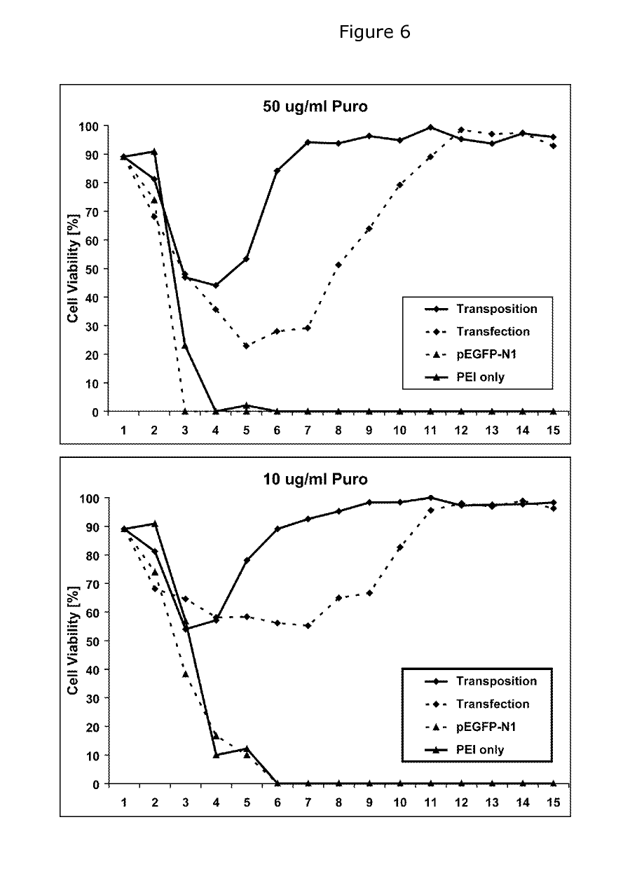

Transposed CHO cell populations expressing a TNFR:Fc fusion protein and the corresponding control populations were generated by cotransfection of CHO-DG44 cells with the appropriate donor and helper plasmids. As negative control cells were transfected either with the pEGFP-N1 vector [SEQ-ID NO:11] (Clontech) which does not contain a puromycin resistance gene or with PEI alone. The day after transfection cells were subjected to puromycin selection at a concentration of either 10 or 50 ug/ml. Cell viability during the whole selection period was assessed using trypan blue exclusion (FIG. 6).

For both selection stringencies (i.e. 10 and 50 ug/ml of puromycin) transposed cell populations showed a similar behaviour with a rapid decrease in the number of viable cells up to 50% 2-3 days after addition of the puromycin selection, subsequently viability started to rapidly increase and reach a percentage of more than 90% after 5-6 days of selection. On the other hand transfected cells showed a dramatic decline in viability when selected with 50 ug/ml and reached the lowest cell viability (ca. 25%) after 4-6 days of selection. Using either 50 or 10 ug/ml of puromycin for selection, transfected cell populations took almost 10 days to fully recover and reach a percentage of viable cells of more than 90%. The faster recovery during selection for transposed cell population may most probably result from an enhanced efficiency of stable transgene integration respectively of stable cell line generation. This property can be used to strongly accelerate the process of cell line development for the production of recombinant proteins.

4.6.3 Determination of the Efficiency of Stable Cell Line Generation

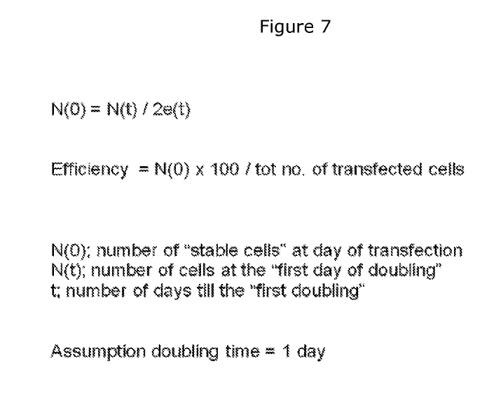

In order to estimate the efficiency of stable cell line generation we, the total number (N(t)) of cells present in the culture the first day in which we could detect a doubling in the total number of viable cells was used to estimated the number of independent clones generated by transposition or transfection. Our method assume that since healthy CHO cells have a doubling time close to 24 h, populations that double cell concentration within a day should consist of almost only stable clones resistant to puromycin. The N(t) value used to determine the number of stable cells generated at the day of transfection and the relative efficiency related to the total number of cells used in transfection, using the formula presented in FIG. 7.

Using this method we could estimate that transposition resulted in stable cell line generation of 8 to 14.6% of the cells used for transfection, whereas efficiencies for conventional transfection were much lower (from 0.5 to 4.6%) (FIG. 8). Our results clearly demonstrate the benefit of using the PB transposon to generate CHO-stable cell lines. Transposition not only increased the number of independent clones that can be generated, but also increased the speed by which the population of stable cells recovers from selection and thus reducing the timeline needed for stable cell line generation.

4.6.4 Piggybac Transposon Mediated Generation of Stable Cell Lines in Absence of Selection

We then tested an alternative approach to validate the improved efficiency of stable cell line generation using the PB system. In this case CHO-DG44 cells were co-transfected with the donor vector pMG-PB-TNFR [SEQ-ID NO:9] along with the helper plasmid pmPBase [SEQ-ID NO:1] at a ratio of 9:1 after which transfected cell populations were cultivated in absence of any selection. The pMG-PB-TNFR [SEQ-ID NO:9] vector carries a bicystronic expression cassette allowing the coordinated expression of a TNFR:Fc fusion protein and eGFP. Using this plasmid an estimation of the efficiency of stable cell line generation will be provided by the percentage of cells in the population still expressing the GFP after a period of time long enough to exclude transiently transfected cells. As control a vector devoid of the PBase gene was used. Percentage and distribution of cells positive for GFP expression was analysed on a daily basis by GUAVA.

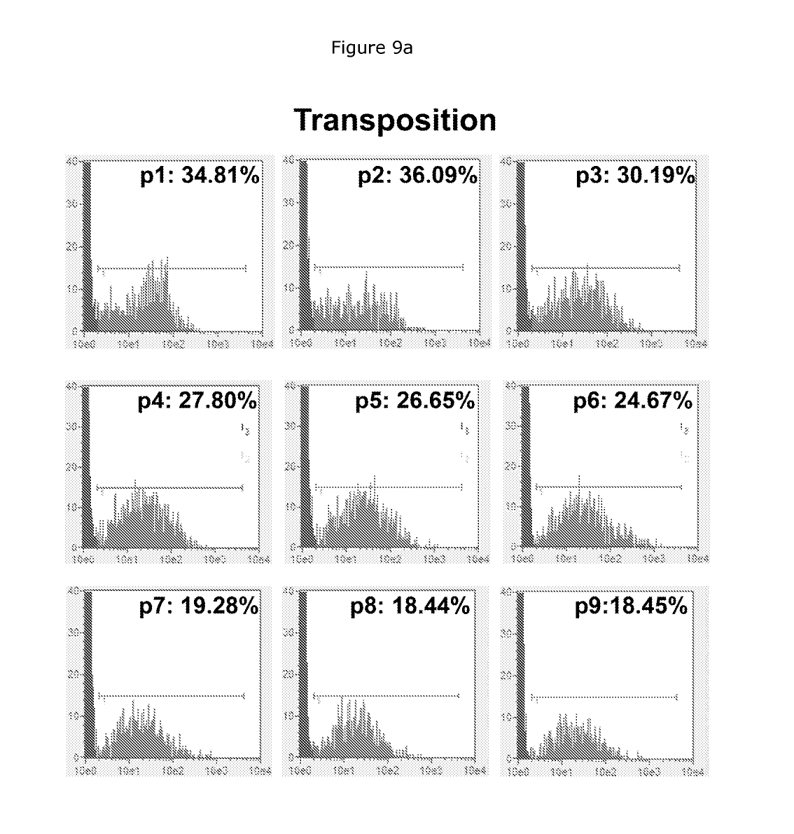

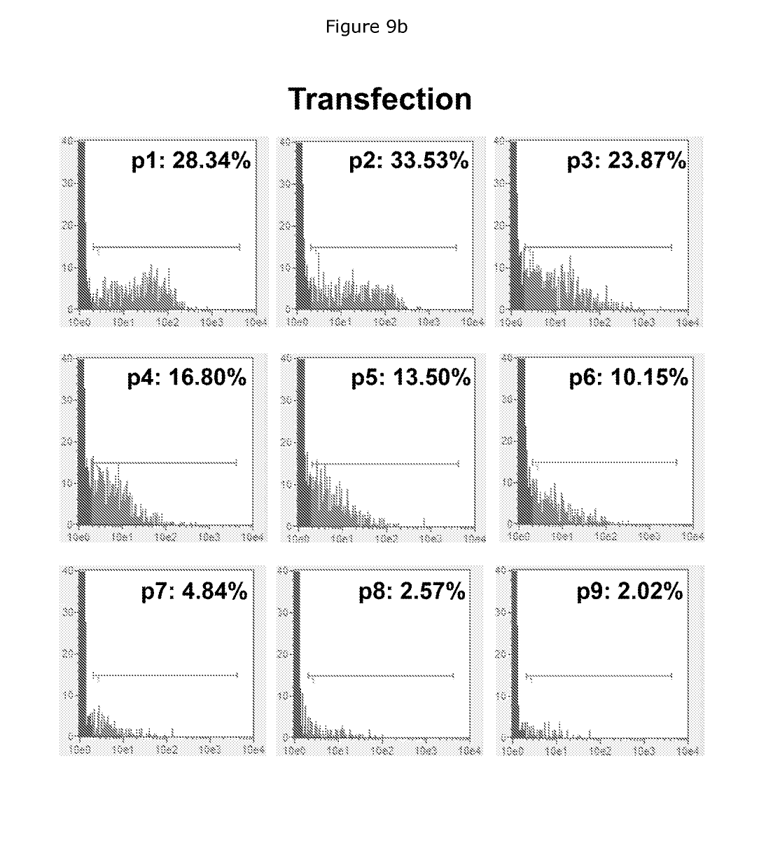

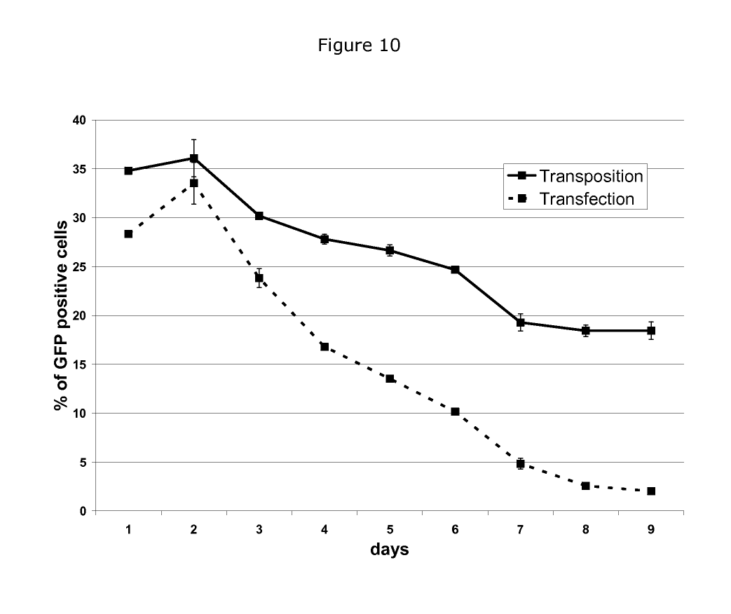

As expected in the transfected cell population the percentage of GFP positive cells rapidly decreased within few days; indeed the efficiency of stable cell line generation upon conventional transfection is known to be a rare event happening in less than 2% of the transfected cells. By contrast more than 18% of the transposed cells were still expressing the GFP, 9 days after transfection furthermore also the level of GFP expression estimated by the mean fluorescence intensity of the positive cells remained constant suggesting that expression might arise from stable integration. In the transposed cell population the initial efficiency of transfection, given by the maximal percentage of GFP positive cells which was reached two days after transfection, was about 36.01%, meaning that more than 50% of cells that actually received the plasmid (i.e. were effectively transfected) stably integrated the transgene. In the absence of the PBase the rate of stable integration relative to transfection efficiency was about 6%. Such numbers might eventually overestimate the real stable integration efficiency since after only 9 days some cells might still express GFP trough a transient mechanism (FIG. 9, FIG. 10).

4.6.5 Cells Lines Generated by Transposition Show an Enhanced Productivity of Recombinant Protein

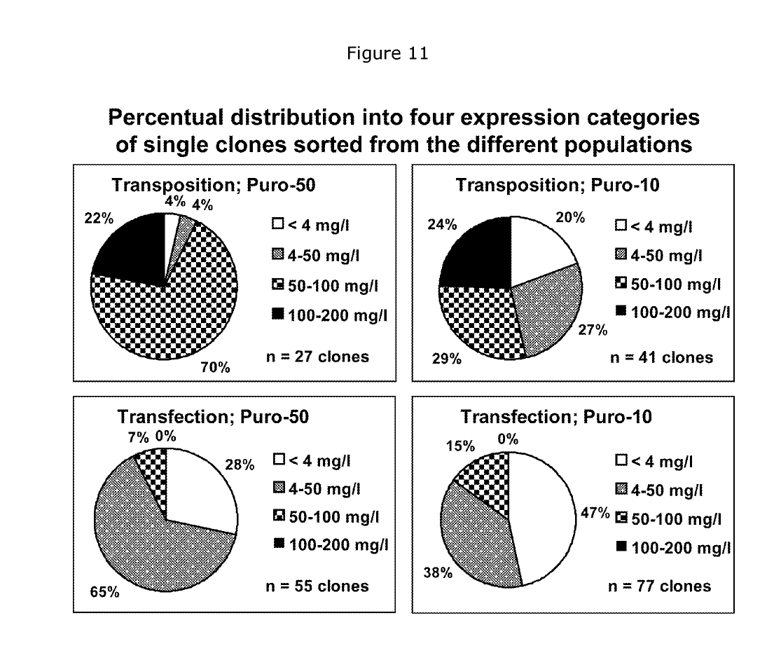

From the cell populations generated after 2 weeks of selection with puromycin either at a concentration of 10 or 50 ug/ml single clones were sorted by limiting dilution. Recovered clonal lines were then passed into 24 wells and screened for TNFR:Fc expression by ELISA. In general we found that clones generated by transposition showed a more than two fold enhancement of the volumetric yields. As summarized in FIG. 6 the majority of the clones generated by transposition had volumetric yields higher than 50 mg/l. By contrast clones sorted by the populations generated by conventional transfection mainly produced less than 50 mg/ml. Increasing the stringency of selection by adding 50 μg/ml of puromycin resulted for both clones generated by transposition or transfection in an enrichment of high expressing clones but had no effect on the maximal yields obtained (see FIG. 11).

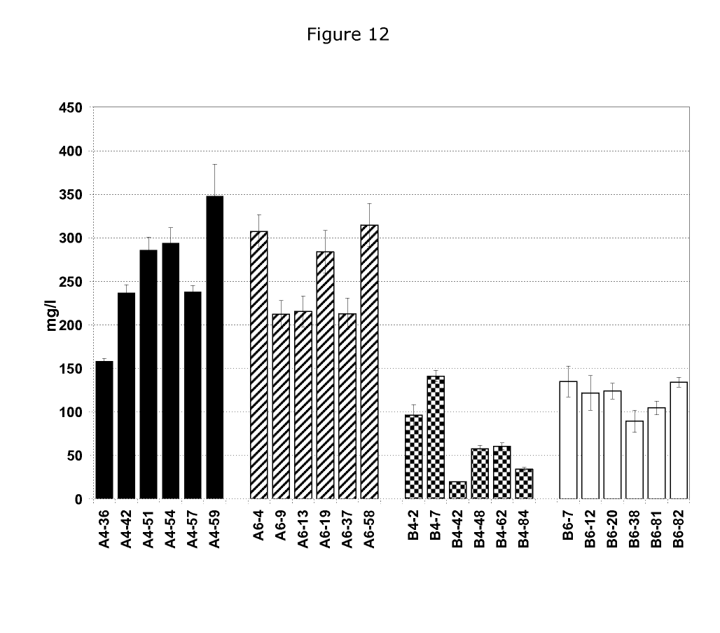

In order to confirm results obtained during the screening process the six best performing clones arising from the four different cell populations (i.e., generated by transposition or transfection and selected with 50 or 10 μg/ml puromycin) were further cultivated and then seeded into TubeSpin bioreactors at an initial concentration of 0.3e6 cells/ml. Supernatant of 4 days old culture was analyzed for TNFR:Fc expression by ELISA. Similar to the results obtained during screening in the 24 well plate format, clones generated by transposition produced the TNFR:Fc recombinant proteins at twofold higher level when compared to clones generated by transfection. Our results demonstrated that transposition not only improves stable transgene integration and thus results in an enhanced number of independent stable clones but also clones generated by transposition usually show increased recombinant protein production (see FIG. 12).

1. A method for creating a stable CHO cell line by co-transfecting a plasmid harboring a piggybac (PB) transposase (PBase) expression cassette with a plasmid harboring a gene expression cassette flanked by piggybac minimal inverted repeat elements where the gene expression cassette flanked by piggybac minimal inverted repeat elements is stably integrated into the CHO genome whereas the plasmid harboring the piggybac transposase is not.

2. A method for creating a pool of stable CHO cell lines by co-transfecting a plasmid harboring a piggybac (PB) transposase (PBase) expression cassette with a plasmid harboring a gene expression cassette flanked by piggybac minimal inverted repeat elements where the gene expression cassette flanked by piggybac minimal inverted repeat elements is stably integrated into the CHO genome whereas the plasmid harboring the piggybac transposase is not.

3. A method for creating a pool of stable CHO cell lines by co-transfecting a plasmid harboring a piggybac (PB) transposase (PBase) expression cassette with a plasmid harboring a gene expression cassette flanked by piggybac minimal inverted repeat elements where a higher number of stable CHO cell clones is obtained with PB transposase co-transfection compared to transfection without PB transposase co-transfection.

4. A method for creating a pool of stable CHO cell lines by co-transfecting a plasmid harboring a piggybac (PB) transposase (PBase) expression cassette with a plasmid harboring a gene expression cassette flanked by piggybac minimal inverted repeat elements where a more rapidly recoverable cell suspension is obtained with PB transposase co-transfection compared to transfection without PB transposase co-transfection.

5. A method for creating a pool of stable CHO cell clones by co-transfecting a plasmid a piggybac (PB) transposase (PBase) expression cassette with a plasmid harboring a gene expression cassette flanked by piggybac minimal inverted repeat elements where the stable pool of cells can be obtained in less than 10 days of selection.

6. A method for creating a pool of stable CHO cell clones by co-transfecting a piggybac (PB) transposase (PBase) expression cassette with a plasmid harboring a gene expression cassette flanked by piggybac minimal inverted repeat elements where the protein of interest is expressed in the clonal culture at higher levels in case of PB transposase co-transfection compared to transfection without PB transposase co-transfection.

7. A method for creating a stable cell line by co-transfecting a plasmid harboring a piggybac (PB) transposase (PBase) expression cassette with a plasmid harboring a gene expression cassette flanked by piggybac minimal inverted repeat elements where the protein of interest is expressed at higher specific productivity in case of PB transposase co-transfection compared to transfection without PB transposase co-transfection.

8. A method for producing a recombinant protein by creating a pool of stable cell clones by means of co-transfecting a plasmid harboring a piggybac (PB) transposase (PBase) expression cassette with a plasmid harboring a gene expression cassette flanked by piggybac minimal inverted repeat elements where the protein of interest is expressed by the pool of clones at higher amounts in case of PB transposase co-transfection compared to transfection without PB transposase co-transfection.

9. The method of claims 1 to 8 wherein said cells are cultivated in the presence of 10 mg/l of puromycin for 10 days.

10. The method of claims 1 to 8 wherein said cells are cultivated in the presence of 10 mg/l of puromycin for less than 10 days.

11. The method of claims 1 to 8 wherein said gene expression cassette comprises the genetic information for a secreted protein.

12. The method of claims 1 to 8 wherein said gene expression cassette comprises the genetic information for a secreted protein and where said secreted protein is produced at a specific productivity of at least 20 pg per cell per day.

13. The method of claims 1 to 8 wherein said gene expression cassette comprises the genetic information for a non-secreted, intracellular or membrane bound protein.
